(19) United States
(12) Plant Patent Application Publication
(10) Pub. No.: US 2018/0184556 P1
(43) Pub. Date: Jun. 28, 2018

(54) Apple tree named 'Gradisca'

(50) ;

(71) Applicants: Olivier Grard, Mudaison (FR); Alexandre Grard, Mudaison (FR)

(72) Inventors: Olivier Grard, Mudaison (FR); Alexandre Grard, Mudaison (FR)

(21) Appl. No.: 15/530,344

(22) Filed: Dec. 27, 2016

(51) Int. Cl. A01H 5/08

(52) U.S. Cl. PLT/168

(57) ABSTRACT

‘Gradisca’ apple tree is the product of an open pollination of ‘Fuji’ apple, and is characterized by its bright red fruit and good production capacity. Fruit of ‘Gradisca’ is sweet and perfumed, and colors well in hot weather. ‘Gradisca’ is distinguished from its parent ‘Fuji’ by its more perfumed fruit and more intense red overcolor.

Latin name: Malus domestica Borkh.

Variety denomination: ‘Gradisca’.

### BACKGROUND OF THE VARIETY

‘Gradisca’ is a new and distinct cultivar of apple tree Malus domestica Borkh discovered in an established ‘Fuji’ orchard. This new cultivar is the product of an open pollination of ‘Fuji’ apple, and is characterized by its bright red fruit and good production capacity. ‘Gradisca’ was first observed in Mudaison, France in 1994. Asexual reproduction by budding and grafting was first carried out at Mudaison in 1996. ‘Gradisca’ has since been observed to remain true to type over successive asexually propagated generations.

### BRIEF DESCRIPTION OF THE VARIETY

‘Gradisca’ was first selected for its attractive bright red fruit and for its productivity. Fruit of ‘Gradisca’ is sweet and perfumed, and colors well in hot weather. There is no equivalent apple with the same organoleptic qualities in the same picking period.

‘Gradisca’ is distinguished from its parent ‘Fuji’ by its more perfumed fruit and more intense red overcolor. Fruit of ‘Gradisca’ is similar in color to that of ‘Rosy Glow’ (not patented), but ‘Gradisca’ is easily distinguished from ‘Rosy Glow’ by its early harvest maturity, at least one month prior to ‘Rosy Glow.’

### BRIEF DESCRIPTION OF THE PHOTOGRAPHS

FIG. 1 shows the leaves of ‘Gradisca’;

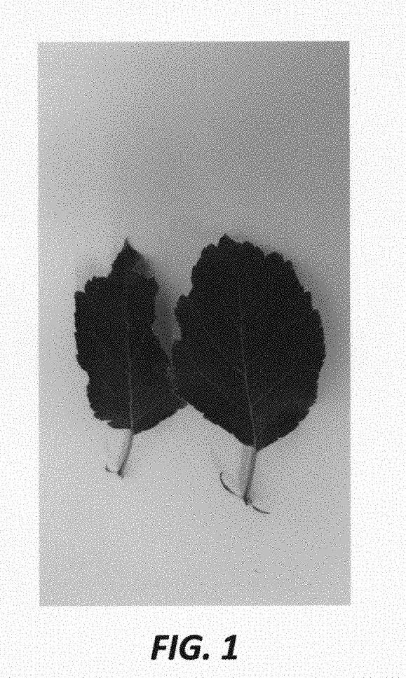

FIG. 2 shows trees of ‘Gradisca’ one week before picking;

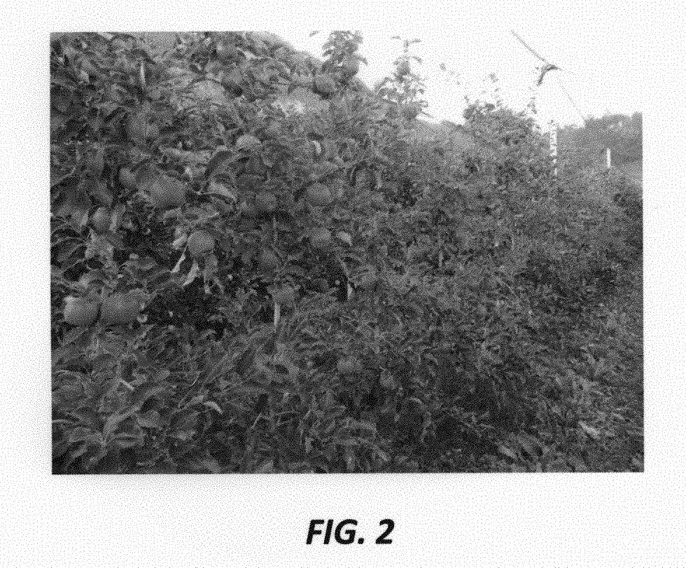

FIG. 3 shows a fruit of ‘Gradisca’;

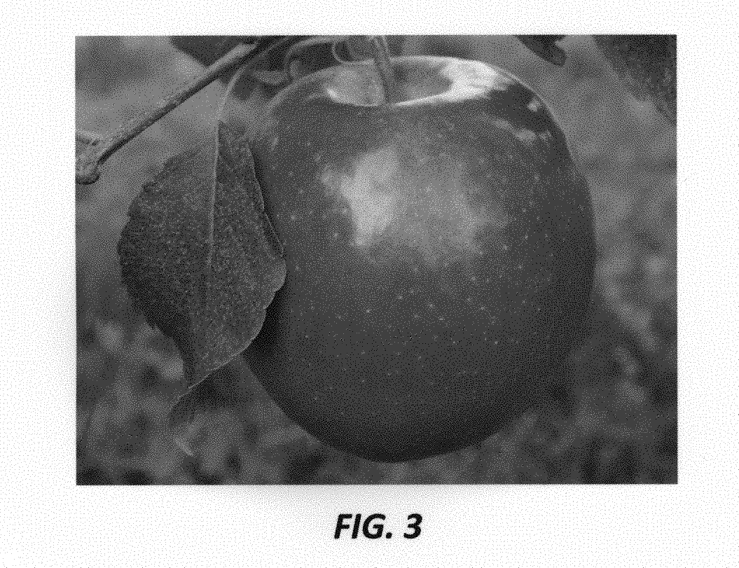

FIG. 4 shows picked whole fruit of ‘Gradisca’; and

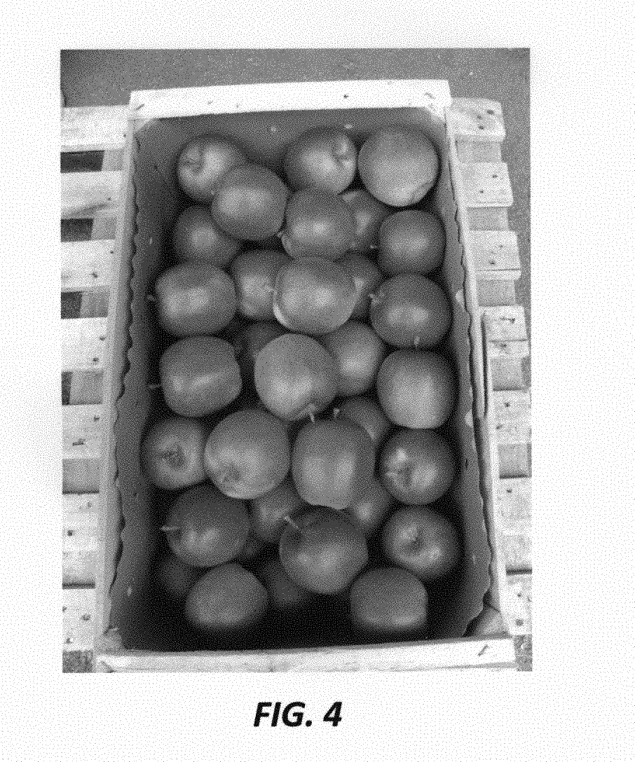

FIG. 5 shows a sectioned fruit of ‘Gradisca.’

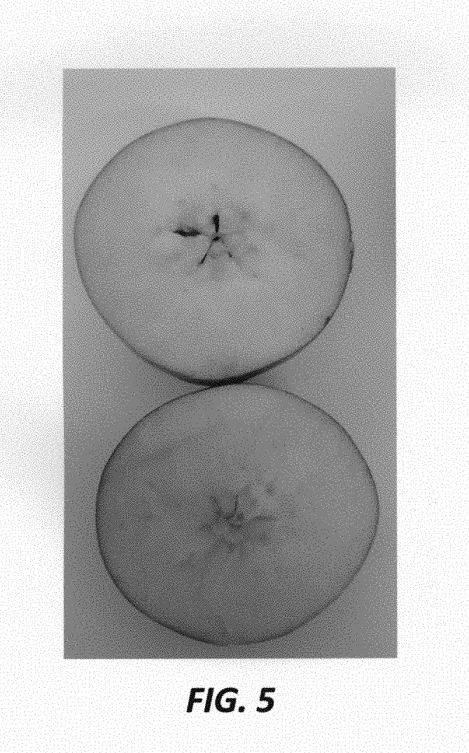

### DETAILED BOTANICAL DESCRIPTION OF THE VARIETY

The following-detailed botanical description is based on observations made during the 2016 growing season at Mudaison, France of four-year-old trees grown on ‘M9 EMLA’ rootstock (not patented). All colors are described according to The Royal Horticultural Society Colour Chart, 5^{th} edition, version 2. It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Vigor.—Vigorous.
   Type.—Ramified, drooping.
   Habit.—Semi-upright tree with one trunk and one leader, main branch spreading.
   Height.—3.20 m.
   Spread.—2 m.
   Trunk diameter.—5.8 cm at 30 cm above the graft union.
   Bark texture.—Fairly smooth with slight crevice and small lenticels.
   Bark coloration.—Light brown 162A.
   Lenticels.—2 mm; light beige 159D.
Branch: (fruiting branches located at around 1 m above the graft union).
   Length.—1.50 m.
   Diameter.—16 mm to 20 mm.
   Crotch angle.—80° to 90°.
   Color.—Dark brown with grey highlights 165B.
   Lenticels.—1.5 mm; beige 159D; 4 to 8 per cm².
One-year-old shoot:
   Length.—25 cm to 35 cm.
   Color.—Very light brown 160A.
   Pubescence.—Strong.
   Thickness.—3 mm to 5 mm.
   Internode length.—13 mm to 22 mm.
   Lenticels.—1 mm; white NN155A; 11 per cm².
Winter hardiness:
   Hardiness.—Hardy in area tested.
   Chilling requirement.—Medium.
Flower buds:
   Quantity per spur.—4 to 5.
   Shape.—Globose.
   Diameter.—10 mm to 12 mm.
   Color.—Red purple 63B.
Flowers:
   Color (balloon stage).—Light pink 36D.
   Diameter of open flower.—2 cm.
   Depth.—0.8 cm.
   Number per cluster.—4.
Petals:
   Quantity per flower.—5.
   Shape.—Ovate.
   Length.—15 mm to 18 mm.
   Width.—14 mm to 16 mm.
   Color of upper surface.—White NN155D.
   Color of lower surface.—White NN155D.
Pistils:
   Length.—12 mm to 13 mm.
   Color.—Yellow-green 145C.
Anthers:
   Quantity.—16.
   Size.—2 mm.
   Pollen.—Present.
   Pollen color.—Yellow 9B.
Stigma:
   Size.—1 mm.
   Color.—Yellow 11C.
Style:
   Size.—8 mm.
   Color.—Yellow-green 145C.
Bloom:
   Date of first bloom.—April 10^{th} (Mudaison, Herault, southern France).
   Date of full bloom.—April 14^{th} (Mudaison, Herault, southern France).
Pedicel:
   Length.—16 mm to 19 mm.
   Diameter.—3 mm.
   Color.—Greyed-orange N172C.
Sepals:
   Quantity.—5.
   Color.—Green 135B.
   Length.—8 mm to 11 mm.
   Shape.—Triangular.
Leaves:
   Shape.—Ovate.
   Length (petiole not included).—7 cm to 9 cm.
   Width.—5 cm to 6 cm.
   Length/width ratio.—1.4 to 1.6.
   Blade margin.—Biserrate.
   Apex.—Acuminate.
   Base shape.—Cuneate.
   Color of top surface.—Green 134A.
   Color of bottom surface.—Green 140B.
   Attitude in relation to shoot.—Outward.
Petiole:
   Length.—18 mm.
   Diameter.—2 mm.
   Color.—Green 142A.
Fruit:
   Quantity per cluster.—4.
   Size.—70 mm to 80 mm diameter.
   Weight.—200 g.
   Ratio of height to width.—0.92.
   General shape in profile.—Globose slightly conical.
   Position of maximum diameter.—At middle.
   Ribbing.—Absent.
   Crowning at calyx end.—Absent or weak.
   Aperture of eye.—Closed.
   Size of eye.—8 mm.
   Length of sepal.—4 mm to 7 mm.
   Depth of eye basin.—9 mm to 13 mm.
   Width of eye basin.—23 mm to 25 mm.
   Length of stalk.—13 mm to 19 mm.
   Thickness of stalk.—4 mm.
   Width of stalk cavity.—17 mm.
   Depth of stalk cavity.—13 mm.
   Size of lenticels.—1.5 mm.
   Bloom of skin.—Absent.
   Greasiness of skin.—Absent.
   Background colour of skin.—Yellow 7A.
   Over colour of skin.—Red-pink N33A.
   Amount of overcolor.—70% to 90%.
   Intensity of overcolor.—Very intense.
   Pattern of overcolor.—Solid flush.
   Flesh texture.—Firm and light.
   Aroma.—Good eating quality, sweet and perfumed.
   Juiciness.—Juicy.
   Brix.—13.5 to 15.5.
   Flesh color.—Yellow 4B.
   Stem color.—Light pink 39D.
Seeds:
   Quantity per fruit.—8 to 10.
   Shape.—Oval.
   Color.—Brown 200D.
Harvest:
   Amount of fruit produced per harvest.—30 kg per tree.
   Harvest date.—September 25 (Mudaison, Herault, southern France).
   Market use.—Fresh.
Diseases/pests:
   Resistance.—None noted.
   Susceptibility.—Susceptible to scab.

We claim:

1. a new and distinct apple tree named ‘Gradisca’ substantially as described and illustrated herein.
